(19) United States
(12) Patent Application Publication
(10) Pub. No.: US 2006/0194761 A1
(43) Pub. Date: Aug. 31, 2006

(54) Olivetol-cyclodextrin complexes and regio-selective process for preparing delta 9-tetrahydrocannabinol

(76) Inventor: Hong Gu, St. Louis, MO (US)

Correspondence Address: Mallinckrodt Inc., 675 McDonnell Boulevard, PO Box 5840, St. Louis, MO, 63134, US

(21) Appl. No.: 10/550,042

(22) PCT Filed: Apr. 2, 2004
(86) PCT No.: PCT/US04/10430

§ 371 Date: Sep. 21, 2005

Related U.S. Application Data
(60) Provisional application No. 60/462,407, filed on Apr. 10, 2003.

(51) Int. Cl. A61K 31/724; C07D 311/80; C08B 37/16

(52) U.S. Cl. 514/58; 549/390; 536/103

(57) ABSTRACT

A cyclodextrin-olivetol derivative complex is provided. The complex effectively blocks reaction at specific carbons to prevent unwanted reactions. A process for preparing a cannabinoid compound is further provided. The process comprises reacting at least one terpenoid with cyclodextrin-olivetol derivative complex to produce the cannabinoid compound.

### FIELD OF THE INVENTION

The present invention relates to the regio-selective synthesis of Δ⁹-hydrodannabinol (THC) and THC derivatives, and more particularly to the condensation reaction of a terpinoid with olivetol and olivetol derivatives using cyclodextrins as space blockers for the regio-selective synthesis of THC.

### BACKGROUND OF THE INVENTION

Naturally occurring cannabinoids are the biologically active components of cannabis. Pharmaceutical interest in cannabinoids has increased due to FDA approval of Δ⁹-tetrahydrocannabinol (THC) for several therapeutic applications

In the 1940's A. R. Todd and R. Adams attempted to prepare several synthetic analogs that were shown to have similar activity of marijuana even before the structure of THC was firmly established. Many efforts have been made to develop an efficient strategy to prepare the THC. Among the several approaches to synthesize THC and its derivatives, the condensation of olivetol with several terpene based compounds, such as (−)-verbenol, (+)-chrysanthanol, (+)-p-mentha-2,8-diene-2-ol, (+)-trans-2-carene epoxide, (+)-3-carene oxide and (+)-p-mentha-2-ene-1,8-diol are more efficient than other approaches, such as Diels-Alder reaction of cinnamic acid derivatives, reaction of citral and lithium derivatives of the olivetol and olivetol dimethyl ether and synthetic route to the THC based on the Pechmann condensation reaction. All known synthesis paths share a common drawback—the final product is a resinous, hard to purify, complex mixture containing up to eight major isomers. As a result, multiple purification steps are often required to purify the THC from the reaction mixture when those synthetic approaches are adopted. Production of THC and THC derivatives is therefor costly to scale up for commercial purposes.

### SUMMARY OF THE INVENTION

An aspect of the present invention is to provide a composition comprising olivetol or an olivetol derivative complexed with at least one cyclodextrin to block unwanted reactions.

Another aspect of the present invention is to provide a process for preparing a cannabinoid compound comprising complexing olivetol or an olivetol derivative with at least one cyclodextrin; and reacting at least one terpenoid with the complexed olivetol to produce the cannabinoid compound.

These are merely illustrative aspects of the present invention and should not be deemed an all-inclusive listing of the innumerable aspects associated with the present invention. These and other aspects will become apparent to those skilled in the art in light of the following disclosure.

### DETAILED DESCRIPTION

A cyclodextrin-olivetol derivative complex is disclosed herein. Cyclodextrins are cyclic oligosaccharides having at least six glucopyranose units. Commercially available cyclodextrins typically have 6, 7 and 8 glucopyranose units. Cyclodextrins are shaped as a torus, with a hydrophilic outer surface and a hydrophobic inner surface. Cyclodextrins are capable of forming inclusion complexes with hydrophobic guest molecules of suitable diameters. These cyclodextrin complexes encapsulate guest molecules.

In the present invention, the cyclodextrin provides its cavity as a non-polar sterically hindered reaction field, in which the olivetol derivative is complexed. In the description below, the term “olivetol derivative: is deemed to include olivetol. The cyclodextrin-olivetol derivative complex is illustrated below.

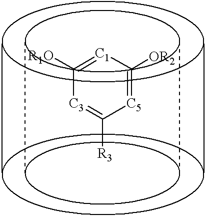

wherein R₁ and R₂ are H or an alkyl group; and wherein R₃ is an akyl having 1 to about 10 carbons, branched or unbranched or an aryl (non-polar). When R₁ and R₂ are H and R₃ is a pentyl group, the compound is olivetol.

In the resulting complex, the C₃ and C₅ positions of the olivetol derivative are blocked, thereby preventing unwanted reactions at these carbons. The C₁ carbon is left unprotected and is available for reaction.

Conventional synthesis of cannabinoids from olivetol derivatives requires a condensation reaction of a substrate with the olivetol derivative at C₁. Reactions at C₃ and C₅ result in unwanted by-products that decrease yield and are difficult to remove.

As a result of the complexation of an olivetol derivative with cyclodextrin, the side reaction pathways related to reactions at the C₃ and C₅ positions have been successfully blocked.

The composition of the cyclodextrin and olivetol derivative non-covalent complex is prepared as an intermediate, which may or may not need to be isolated for further reaction to prepare THC.

The reaction may be carried out in a one or two-step process. For the two-step reaction process, the cyclodextrin-olivetol derivative complex is isolated, and then converted to the desired product at a later time.

The selection of a suitable cyclodextrin depends primarily on the sizing of the non-polar cavity. Cyclodextrins suitable for complexation with olivetol derivatives include but are not limited to natural α-cyclodextrin, β-cyclodextrin, γ-cyclodextrin or modified synthetic cyclodextrin, such as (2-hydroxy-propyl)-β-cyclodextrin, (2-carboxyethyl)-α,β,γ-cyclodextrin, (2,6-Di-O)-ethyl-β-cyclodextrin and (2-hydroxy-ethyl)-β-cyclodextrin.

The cyclodextrin-olivetol derivative complex is formed by mixing the cyclodextrin and olivetol derivative in a suitable solvent. Suitable solvents include but are not limited to tetrahydrofuran, dimethyl-formaldehyde, hydrocarbons, halogenated hydrocarbons, ethers such as diethyl ether, ketones such as acetone and methyl ethyl ketone, alcohols such as methanol, ethanol and isopropyl alcohol and mixtures thereof. Preferred solvents include halogenated hydrocarbons, tetrahydrofuran and dimethyl formaldehyde. The reaction is preferably at room temperature for about 30 minutes, although time and temperature are not critical. The solvent is then evaporated at reduced pressure, leaving a solid cyclodextrin-olivetol derivative complex.

The reaction of the cyclodextrin-olivetol derivative complex is illustrated below:

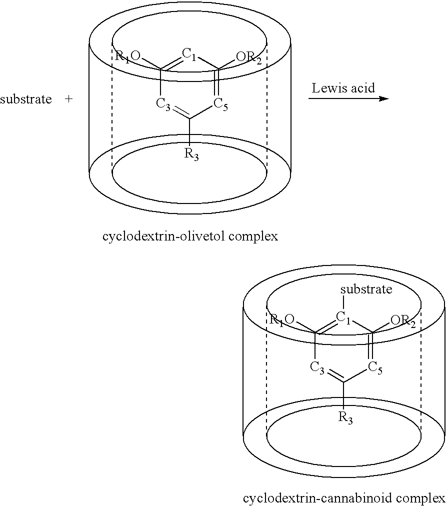

To prepare THC cannabinoids, the substrates used in this reaction include (−)-verbenol, (+)-chrysanthanol, (+)-p-mentha-2,8-diene-2-ol, (+)-trans-2-carene epoxide, (+)-3-carene oxide and (+)-p-mentha-2-ene-1,8-diol. These substrates are illustrative and are not meant to be limiting of the present invention.

The preparation of a THC derivative from an olivetol derivative is well known in the art. The process includes dissolving the cyclodextrin-olivetol derivative complex in a solvent system as defined above. While maintaining a reduced temperature, the substrate and an acid catalyst, including but not limited to Lewis acids, are added to the cyclodextrin-olivetol derivative complex. The temperature is typically maintained at about 0° C. to about 15° C., with about 5° C. being preferred. The reaction process may be monitored with HPLC, and upon completion of the reaction the reaction may be quenched with a base. The resulting mixture is purified by conventional methods known in the art.

In addition, the above reaction may be altered to result in the formation of a cannabidiol, typically by using a weaker acid catalyst or by reducing the temperature of the reaction, as is well known in the art. The cannabidiol can then be converted to a cannabinoid compound or utilized as an intermediate for a different reaction.

Futhermore, the presence of ABN-cannabidiol has been detected in the reaction mixture, the ABN-cannabidiol being the result of either reaction of the (+)-2,8-menthadiene-1-ol at the C₃ or C₅ position due to incomplete complexation of the cyclodextrin/olivetol, or the result of rearrangement of the normal cannabidiol. In either case, it has been determined the ABN-cannabidiol, in the presence of at least one cylcodextrin and at least one Lewis acid, rearranges to normal cannabidiol.

The following examples are offered to illustrate aspects of the present invention, and are not intended to limit or define the present invention in any manner

### EXAMPLES

### Example 1

### The preparation of 5-pentyl-1,3-benzenediol/cyclodextrin complex

5 g of olivetol and 31 g of β-cyclodextrin were mixed in 500 ml tetrahedronfuran and stirred at 25° C. for about 30 minutes. The solvent was evaporated at reduced pressure. A white solid of the 5-pentyl-1,3-benzenediol/cyclodextrin complex, about 36 g, was obtained.

### Example 2

### Preparation of (−)-2-(p-mentha-2,8-diene-3-yl)pentylbenzene-1,3-diol

The freshly prepared olivetol/cyclodextrin complex of Example 1 and 9 g of MgSO₄ were mixed together and stirred in 500 ml of tetrahedronfuran. The reaction mixture was cooled in an ice water bath to keep the temperature at about 5° C. 4.4 g of (+)-2,8-menthadiene-1-ol was placed in an addition funnel and p-TSA acid was placed into a syringe. The (+)-2,8-menthadiene-1-ol and the acid catalyst were added to the reaction mixture drop wise over 15 minutes. The reaction progress was monitored by HPLC and, upon completion of the reaction, an excess of NaHCO₃ was added to quench the reaction.

Salts were filtered out from the reaction mixture and the organic solvent was evaporated, leaving about 7.5 g of an oil. The oil was dissolved into 100 ml of petroleum ether and was washed with 300 ml of water twice and brine solution once. The product mixture was purified via chromatography on a silica gel column utilizing heptane/acetonitrile (98:2) as the mobile phase. A fraction contained the (−)-cannabidiol, also known as (−)-2-(p-mentha-2,8-diene-3yl)pentylbenzene-1,3-diol, which was concentrated to give an oil.

¹H NMR δH (300 MHz, CHCl3): 0.89 (3H,t), 1.27 (4H, m), 1.56 (2H,m), 1.65 (3H,s), 1.79 (3H,s), 2.11 (2H,m), 2.44 (3H, m), 3.85 (1H,d), 4.6 (2H,d), 5.58 (1H,s), 6.22 (2H,s). ¹³C NMR δH (300 mHz, CHCl3): 14.6, 20.8, 23.3, 24.3, 28.7, 30.8, 37.4, 45.6, 108.2, 110.0, 111.4, 111.6, 124.2, 140.7, 143.5, 145.4, 156.3.

### Example 3

### Preparation of (−)-trans-Δ⁹-tetrahydrocannabinol

The freshly prepared olivetol/cyclodextrin complex of Example 1 and 9 g of MgSO₄ were mixed together in 500 ml of tetrahydrofuran. The reaction mixture was cooled in an ice water bath to keep the temperature at about 5° C. 4.4 g of (+)-2,8-menthadiene-1-ol was placed in an addition funnel and BF₃Et₂O acid was placed into a syringe. The (+)-2,8-menthadiene-1-ol and the acid catalyst were added to the reaction mixture drop wise over 15 minutes. The reaction progress was monitored by HPLC and, upon completion of the reaction, an excess of NaHCO₃ was added to quench the reaction. Salts were filtered out from the reaction mixture and the organic solvent was evaporated to give an oil. Approximately 7.0 g of the oil was obtained as a mixture of (−)-trans-Δ⁹-tetrahydrocannabinol and some minor amount of (−)-trans-Δ⁸-tetrahydrocannabinol. The oil was dissolved into 100 ml of petroleum ether and was washed with 300 ml of water twice and brine solution once. The product mixture was purified via chromatography on a silica gel column and (−)-trans-Δ⁹-tetrahydrocannabinol eluted with heptane/acetonitrile (98:2) as mobile phase. A fraction containing the (−)-trans-Δ⁹-tetrahydrocannabinol, with purity over 98%, was concentrated to give a light yellow oil.

Having described the invention in detail, those skilled in the art will appreciate that modifications may be made of the invention without departing from its spirit and scope. Therefore, it is not intended that the scope of the invention be limited to the specific embodiments described. Rather, it is intended that the appended claims and their equivalents determine the scope of the invention.

1. A composition comprising an olivetol derivative complexed with at least one cyclodextrin.

2. The composition according to claim 1 wherein the at least one cyclodextrin includes a cyclodextrin selected from the group consisting of natural α-cyclodextrin, β-cyclodextrin, γ-cyclodextrin or modified synthetic cyclodextrin, such as (2-hydroxy-propyl)-β-cyclodextrin, (2-carboxyethyl)-α,β,γ-cyclodextrin, (2,6-Di-O)-ethyl-β-cyclodextrin and (2-hydroxy-ethyl)-β-cyclodextrin.

3. The composition according to claim 1 wherein the olivetol derivative comprises 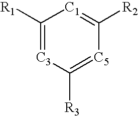
wherein R₁ and R₂ are H or an alkyl or alcohol; and wherein R₃ is selected from the group consisting of normal akyl groups having 1 to about 10 carbons, branched alkyl groups having 1 to about 10 carbons and aryl groups.

4. The composition according to claim 1 wherein the olivetol derivative is olivetol.

5. (canceled)

6. A process for preparing a cannabinoid compound comprising:
complexing an olivetol derivative with at least one cyclodextrin; and
reacting at least one terpenoid with the complexed olivetol derivative to produce the cannabinoid compound.

7. The process according to claim 6 wherein the at least one cyclodextrin includes a cyclodextrin selected from the group consisting of natural α-cyclodextrin, β-cyclodextrin, γ-cyclodextrin or modified synthetic cyclodextrin, such as (2-hydroxy-propyl)-β-cyclodextrin, (2-carboxyethyl)-α,β,γ-cyclodextrin, (2,6-Di-O)-ethyl-β-cyclodextrin and (2-hydroxy-ethyl)-β-cyclodextrin.

8. The process according to claim 6 wherein the at least one terpenoid is selected from the group consisting of (−)-verbenol, (+)-chrysanthanol, (+)-p-mentha-2,8-diene-2-ol, (+)-trans-2-carene epoxide, (+)-3-carene oxide and (+)-p-mentha-2-ene-1,8-diol.

9. The process according to claim 6 further including maintaining a temperature below room temperature while reacting the at least one terpenoid with the complexed olivetol derivative.

10. The process according to claim 9 wherein the temperature is about 0° C. to about 15° C.

11. The process according to claim 6 further including adding at least one acid catalyst.

12. The process according to claim 6 further including quenching the reaction of the at least one terpenoid with the complexed olivetol derivative with a base.

13. The process according to claim 6 wherein the cannabinoid is a naturally occurring component of cannabis.

14. The process according to claim 6 wherein the cannabinoid is a synthetic analog of cannabis.

15. A process for preparing a cannabidiol compound comprising:
complexing an olivetol derivative with at least one cyclodextrin; and
reacting at least one terpenoid with the complexed olivetol derivative at a temperature low enough to result in the production of a cannabidiol compound.

16. The process according to claim 15 wherein the at least one cyclodextrin includes a cyclodextrin selected from the group consisting of natural α-cyclodextrin, β-cyclodextrin, γ-cyclodextrin or modified synthetic cyclodextrin, such as (2-hydroxy-propyl)-β-cyclodextrin, (2-carboxyethyl)-α,β,γ-cyclodextrin, (2,6-Di-O)-ethyl-β-cyclodextrin and (2-hydroxy-ethyl)-β-cyclodextrin.

17. The process according to claim 15 wherein the at least one terpenoid is selected from the group consisting of (−)-verbenol, (+)-chrysanthanol, (+)-p-mentha-2,8-diene-2-ol, (+)-trans-2-carene epoxide, (+)-3-carene oxide and (+)-p-mentha-2-ene-1,8-diol.

18. The process according to claim 15 further including adding at least one acid catalyst while reacting the at least one terpenoid with the complexed olivetol derivative, wherein the acid catalyst is selected to result in the formation of the cannabidiol.

19. The process according to claim 15 further including quenching the reaction of the at least one terpenoid with the complexed olivetol derivative with a base.

20. A process for preparing Δ⁹-tetrahydrocannabinol comprising:
complexing olivetol with at least one cyclodextrin; and
reacting the complexed olivetol with (+)-p-mentha-2,8-diene-1-ol to form Δ⁹-tetrahydrocannabinol.

21. The process according to claim 20 wherein the at least one cyclodextrin includes a cyclodextrin selected from the group consisting of natural α-cyclodextrin, β-cyclodextrin, γ-cyclodextrin or modified synthetic cyclodextrin, such as (2-hydroxy-propyl)-β-cyclodextrin, (2-carboxyethyl)-α,β,γ-cyclodextrin, (2,6-Di-O)-ethyl-β-cyclodextrin and (2-hydroxy-ethyl)-β-cyclodextrin.

22. The process according to claim 20 further including maintaining a temperature below room temperature while reacting the (+)-p-mentha-2,8-diene-1-ol with the complexed olivetol.

23. The process according to claim 20 wherein the temperature is about 0° C. to about 15° C.

24. The process according to claim 20 further including adding at least one acid catalyst while reacting the (+)-p-mentha-2,8-diene-1-ol with the complexed olivetol.

25. The process according to claim 20 further including quenching with the reaction of the (+)-p-mentha-2,8-diene-1-ol with the complexed olivetol with a base.

26-30. (canceled)
